(19) United States
(12) Patent Application Publication
(10) Pub. No.: US 2022/0354705 A1
(43) Pub. Date: Nov. 10, 2022

(54) NO SLIP COMPRESSION KNEE SLEEVE

(71) Applicant: Thomas Christopher Wells, Wichita, KS (US)

(72) Inventor: Thomas Christopher Wells, Wichita, KS (US)

(21) Appl. No.: 17/314,086

(22) Filed: May 7, 2021

(51) Int. Cl. A61F 13/08; A41B 9/14; A41B 9/08; A41B 9/00

(52) U.S. Cl. CPC A61F 13/08; A41B 9/14; A41B 9/08; A41B 9/001

(57) ABSTRACT

An orthotic device with an integrated compression knee sleeve that is less prone to slippage includes a waistband configured to encircle a wearer's waist; a seat portion and a front portion attached below the waistband to encircle the wearer's seat and crotch; a first leg portion attached below the seat portion and the front portion to partially cover a first leg of the wearer; and a second leg portion attached below the seat portion and the front portion to partially cover a second leg of the wearer. The second leg portion includes the integral compression knee sleeve, which encircles the wearer's knee to exert compressive forces on the knee.

### BACKGROUND

Compression knee sleeves are often worn by athletes and others to reduce knee pain and to resist injuries. Such sleeves increase knee warmth and blood flow, limit patella movement, and increase proprioception. Most compression knee sleeves are made of highly compressive materials and are designed to slide over the top of a wearer's knee. Unfortunately, such knee sleeves are prone to slipping down the wearer's leg, especially during vigorous activities. To limit undesired slippage, Velcro straps, rubber bands, belts, and the like may be wrapped around the tops of the sleeves, but such tightening mechanisms are uncomfortable, add undesired weight, and often inhibit leg mobility.

### SUMMARY

The present invention solves the above-described problems and provides a distinct advance in the art of compression knee sleeves by providing an orthotic device with an integrated compression knee sleeve that is less prone to slippage without requiring Velcro straps, rubber bands, belts, or other tightening mechanisms.

An embodiment of the orthotic device broadly comprises a waistband configured to encircle a wearer's waist; a seat portion and a front portion attached below the waistband to encircle the wearer's seat and crotch; a first leg portion attached below the seat portion and the front portion to partially cover a first leg of the wearer; and a second leg portion attached below the seat portion and the front portion to partially cover a second leg of the wearer.

In accordance with an important aspect of the invention, the second leg portion includes an integral compression knee sleeve that encircles the wearer's knee to exert compressive forces on the knee. The waistband, seat portion, and front portion exert an upward anchoring force on the compression knee sleeve to prevent it from slipping downward on the wearer's leg. In some embodiments, the orthotic device may also comprise a stirrup that exerts a downward anchoring force on the compression knee sleeve to prevent it from creeping up the wearer's leg. Thus, the waistband, seat portion, and front portion and the stirrup cooperatively maintain optimal centering of the compression knee sleeve over the wearer's knee.

In one embodiment, the waistband, seat portion, front portion, and first leg portion are formed of a first material such as nylon, lycra, polyester, spandex, or a blend of these materials and/or other materials, and the compression knee sleeve is formed of a second material that is thicker and/or more compressive than the first material such as neoprene or a blend of neoprene and nylon, lycra, polyester, or spandex. The waistband, seat portion, and front portion can thus be worn in a looser fashion while the compression knee sleeve can be worn more tightly to provide the desired compressive forces on the wearer's knee.

To further prevent the compression knee sleeve from slipping down or creeping up when the waistband is pulled up or down, some embodiments of the orthotic device may also comprise a relief band above the compression knee sleeve. The relief band stretches or otherwise expands when the waistband is pulled up and contracts when the waistband is pulled down so as to minimize upward and downward forces on the compression knee sleeve as the waistband moves. In one embodiment, the relief band includes accordion-shaped folds and is formed of a material that is thinner than the material of the compression knee sleeve.

Because the orthotic device fits tightly on the wearer and resists downward slippage and upward creeping of the compression knee sleeve, it may be worn under a knee brace without causing unwanted movement of the knee brace. The orthotic device may also be worn without a knee brace.

This summary is provided to introduce a selection of concepts in a simplified form that are further described in the detailed description below. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Embodiments of the present invention are described in detail below with reference to the attached drawing figures, wherein:

FIG. 1 is a partial perspective view of an orthotic device constructed in accordance with an embodiment of the present invention shown on a wearer.

FIG. 2 is a front view of the orthotic device.

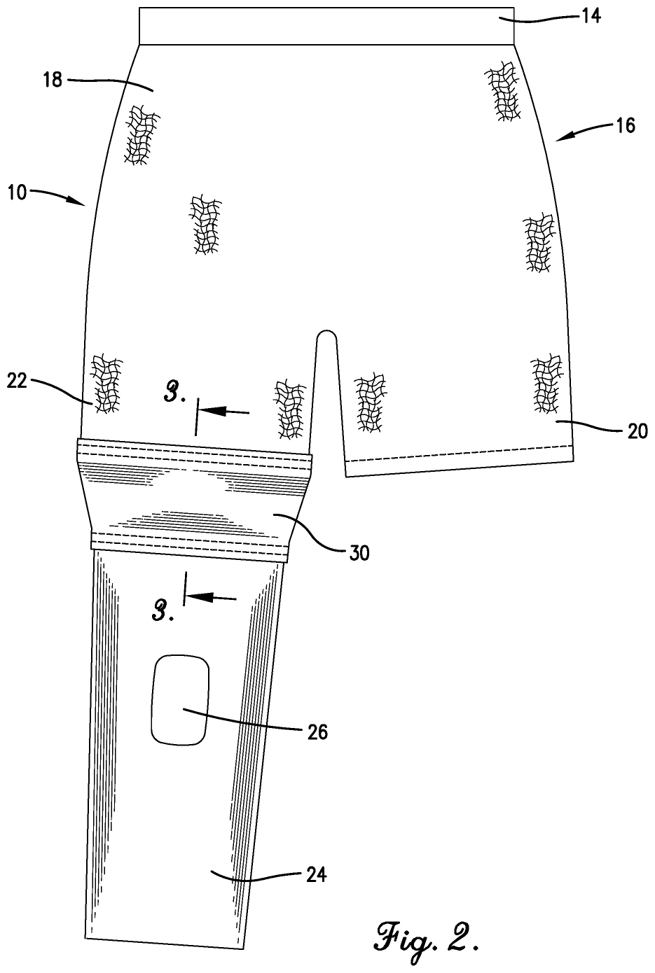

FIG. 3 is a partial vertical sectional view of the orthotic device taken along line 3-3 of FIG. 2.

FIG. 4 is a partial perspective view of an orthotic device constructed in accordance with another embodiment of the present invention shown on a wearer.

The drawing figures do not limit the present invention to the specific embodiments disclosed and described herein. The drawings are not necessarily to scale, emphasis instead being placed upon clearly illustrating the principles of the invention.

### DETAILED DESCRIPTION

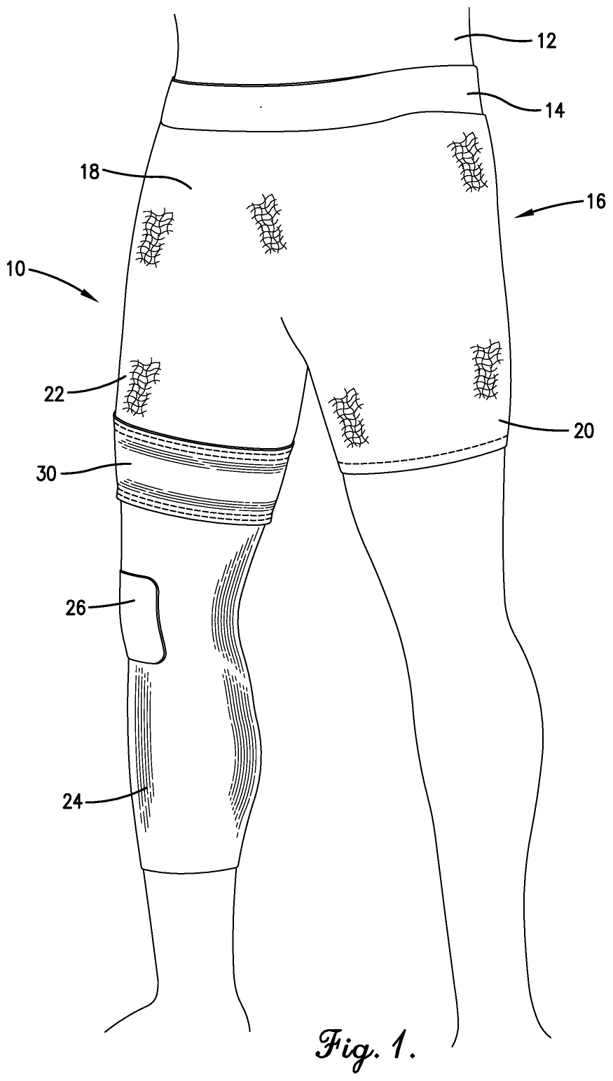

Turning now to the drawing figures, and particularly FIG. 1, an orthotic device 10 constructed in accordance with an embodiment of the invention is shown worn by an athlete or other wearer 12. As discussed in more detail below, the orthotic device includes an integrated compression knee sleeve 24 that is less prone to slippage without requiring Velcro straps, rubber bands, belts, or other tightening mechanisms. Because the orthotic device fits tightly on the wearer and resists downward slippage and upward creeping of the compression knee sleeve, it may be worn under a knee brace without causing unwanted movement of the knee brace. The orthotic device may also be worn without a knee brace.

An embodiment of the orthotic device broadly comprises a waistband 14; a seat portion 16; a front portion 18; a first leg portion 20; and a second leg portion 22. The second leg portion 22 includes the integral compression knee sleeve 24, but the knee sleeve may alternatively be in the first leg portion, or a knee sleeve may be integrated in both leg portions.

The waistband 14 is configured to encircle a wearer's waist and may be formed of a first material such as nylon, lycra, polyester, spandex, or a blend of these materials and/or other materials. Laces, or other closure mechanisms may be formed in the waistband, but in preferred forms, the waistband is held in place on the wearer by compressive forces only.

The seat portion 16 and front portion 18 are attached below the waistband to encircle the wearer's seat and crotch and are preferably made of the same materials as the waistband. The front portion may have a zippered fly opening or other opening for versions of the orthotic device designed for men.

The first leg portion 20 is attached below the seat portion 16 and the front portion 18 to cover a first leg of the wearer at least partially. The first leg portion may be thigh-length as illustrated, may be longer to cover the wearer's entire leg, or may be of any other length. Although the first leg portion is shown on the wearer's left leg, the positioning of the first and second leg portions may be reversed so that the first leg portion is on the wearer's right leg.

The second leg portion 22 is also attached below the seat portion and the front portion to partially cover a second leg of the wearer. In accordance with an important aspect of the invention, the compression knee sleeve 24 is integrated in the second leg proton to encircle the wearer's knee so as to exert compressive forces on the knee.

The compression knee sleeve 24 is formed of a second material that is thicker and/or more compressive than the first material. The waistband, seat portion, and front portion can thus be worn in a looser fashion while the compression knee sleeve can be worn more tightly to provide the desired compressive forces on the wearer's knee.

In some embodiments, the knee sleeve 24 is formed of neoprene or a blend of neoprene and nylon, lycra, polyester, or spandex. In some embodiments, the compression knee sleeve may be formed of neoprene infused with copper. The compression knee sleeve may also have an anterior knee-cap covering patch 26 that adds more compressive forces directly over the wearer's knee.

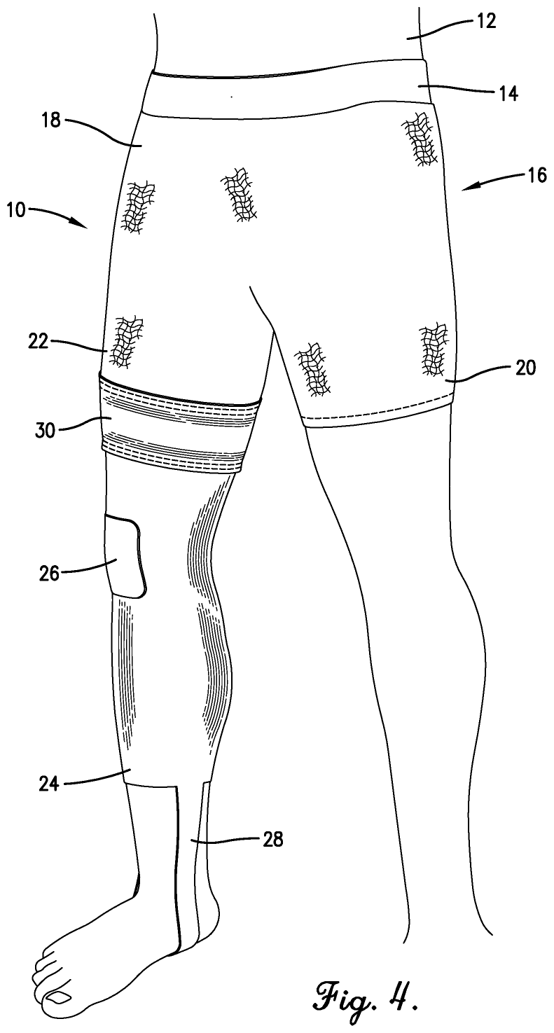

As best shown in FIG. 4, another embodiment of the orthotic device may also comprise a stirrup 28 attached below the compression knee sleeve for positioning under one of the wearer's feet to prevent the compression knee sleeve from creeping up the wearer's leg. In other embodiments, the lowest margin of the compression knee sleeve may include an elastic band or other mechanism for resisting upward creeping rather than the stirrup.

Because the compression knee sleeve 24 is integrated in the orthotic device, it does not slip up or creep down the wearer's leg. The waistband 14, seat portion 16, and front portion 18 exert an upward anchoring force on the compression knee sleeve that prevents downward slippage of the knee sleeve while the stirrup 28 exerts a downward anchoring force on the compression knee sleeve to prevent it from creeping up the wearer's leg. Thus, the waistband, seat portion, and front portion and the stirrup cooperatively maintain optimal centering of the compression knee sleeve over the wearer's knee.

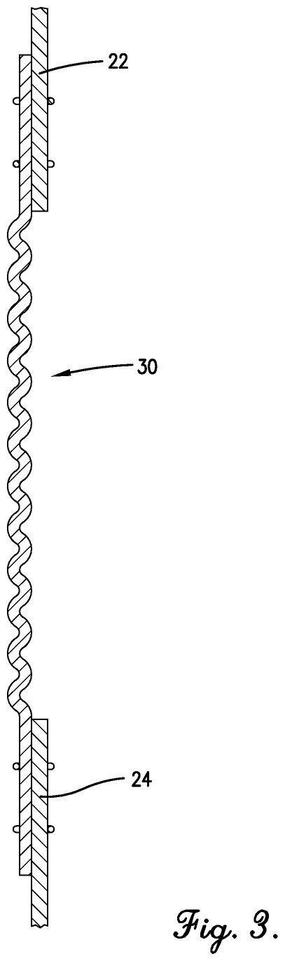

In some embodiments of the invention, the orthotic device may further comprise a relief band 30 positioned in the second leg portion above the compression knee sleeve 24. The relief band 30 stretches or otherwise expands when the waistband is pulled up and contracts when the waistband is pulled down so as to minimize upward and downward forces on the compression knee sleeve as the waistband moves so as to help prevent the compression knee sleeve from slipping down or creeping up when the waistband is pulled up or down. As shown in FIG. 3, an embodiment of the relief band includes accordion-shaped folds and is thinner and/or lighter than the second material. In other embodiments of the orthotic device, the relief band is omitted, and the second leg portion transitions seamlessly into the compression knee sleeve.

Although the drawing figures depict only one compression knee sleeve 24, other embodiments may include a compression knee sleeve in both the first and second leg portions.

In this description, references to “one embodiment,” “an embodiment,” or “embodiments” mean that the feature or features being referred to are included in at least one embodiment of the technology. Separate references to “one embodiment,” “an embodiment,” or “embodiments” in this description do not necessarily refer to the same embodiment and are also not mutually exclusive unless so stated and/or except as will be readily apparent to those skilled in the art from the description. For example, a feature, structure, act, etc. described in one embodiment may also be included in other embodiments but is not necessarily included. Thus, the current technology can include a variety of combinations and/or integrations of the embodiments described herein.

Although the present application sets forth a detailed description of numerous different embodiments, the legal scope of the description is defined by the words of the claims set forth at the end of this patent and equivalents. The detailed description is to be construed as exemplary only and does not describe every possible embodiment since describing every possible embodiment would be impractical. Numerous alternative embodiments may be implemented, using either current technology or technology developed after the filing date of this patent, which would still fall within the scope of the claims.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The patent claims at the end of this patent application are not intended to be construed under 35 U.S.C. § 112(f) unless traditional means-plus-function language is expressly recited, such as “means for” or “step for” language being explicitly recited in the claim(s).

Although the invention has been described with reference to the embodiments illustrated in the attached drawing figures, it is noted that equivalents may be employed and substitutions made herein without departing from the scope of the invention as recited in the claims.

Having thus described various embodiments of the invention, what is claimed as new and desired to be protected by Letters Patent includes the following:

1. An orthotic device comprising:
a waistband configured to encircle a wearer's waist;
a seat portion and a front portion attached below the waistband to encircle the wearer's seat and crotch;
a first leg portion attached below the seat portion and the front portion to partially cover a first leg of the wearer; and
a second leg portion attached below the seat portion and the front portion to partially cover a second leg of the wearer, the second leg portion including an integral compression knee sleeve that encircles a knee of the second leg to exert compressive forces on the knee.

2. The orthotic device as set forth in claim 1, wherein the waistband, the seat portion, the front portion, and the first leg portion are formed of a first material, and the compression knee sleeve is formed of a second material that is thicker and more compressive than the first material.

3. The orthotic device as set forth in claim 2, wherein the first material comprises nylon, lycra, polyester, or spandex.

4. The orthotic device as set forth in claim 2, wherein the first material comprises a blend of nylon, lycra, polyester, or spandex.

5. The orthotic device as set forth in claim 2, wherein the second material is neoprene.

6. The orthotic device as set forth in claim 2, wherein the second material is a blend of neoprene and nylon, lycra, polyester, or spandex.

7. The orthotic device as set forth in claim 6, wherein the second material is infused with copper.

8. The orthotic device as set forth in claim 2, further comprising a relief band positioned in the first leg portion above the compression knee sleeve.

9. The orthotic device as set forth in claim 8, wherein the relief band is formed of a third material that is thinner and weaker than the second material.

10. The orthotic device as set forth in claim 1, further comprising a stirrup attached below the compression knee sleeve for positioning under one of the wearer's feet to prevent the compression knee sleeve from creeping up the wearer's knee.

11. An orthotic device comprising:
a waistband configured to encircle a wearer's waist;
a seat portion and a front portion attached below the waistband to encircle the wearer's seat and crotch, the seat portion and the front portion formed of a first material;
a first leg portion attached below the seat portion and the front portion to partially cover a first leg of the wearer, the first leg portion formed of the first material;
a second leg portion attached below the seat portion and the front portion to partially cover a second leg of the wearer, the second leg portion including an integral compression knee sleeve that encircles a knee of the second leg to exert compressive forces on the knee, the compression knee sleeve formed of a second material that is thicker and more compressive than the first material; and
a relief band positioned in the first leg portion above the compression knee sleeve, the relief band formed of a third material.

12. The orthotic device as set forth in claim 11, wherein the third material is thinner and weaker than the second material.

13. The orthotic device as set forth in claim 11, wherein the first material comprises nylon, lycra, polyester, or spandex.

14. The orthotic device as set forth in claim 11, wherein the first material comprises a blend of nylon, lycra, polyester, or spandex.

15. The orthotic device as set forth in claim 11, wherein the second material is neoprene.

16. The orthotic device as set forth in claim 11, wherein the second material is a blend of neoprene and nylon, lycra, polyester, or spandex.

17. The orthotic device as set forth in claim 11, wherein the second material is infused with copper.

18. The orthotic device as set forth in claim 11, further comprising a stirrup attached below the compression knee sleeve for positioning under one of the wearer's feet to prevent the compression knee sleeve from creeping up the wearer's knee.

19. An orthotic device comprising:
a waistband configured to encircle a wearer's waist;
a seat portion and a front portion attached below the waistband to encircle the wearer's seat and crotch, the seat portion and the front portion formed of a first material;
a first leg portion attached below the seat portion and the front portion to partially cover a first leg of the wearer, the first leg portion formed of the first material and terminating on a thigh of the first leg;
a second leg portion attached below the seat portion and the front portion to partially cover a second leg of the wearer, the second leg portion including an integral compression knee sleeve that encircles a knee of the second leg to exert compressive forces on the knee, the compression knee sleeve formed of a second material that is thicker and more compressive than the first material;
a relief band positioned in the first leg portion above the compression knee sleeve, the relief band formed of a third material that is thinner and weaker than the second material; and
a stirrup attached below the compression knee sleeve for positioning under one of the wearer's feet to prevent the compression knee sleeve from creeping up the wearer's knee.

20. The orthotic device as set forth in claim 19, wherein the first material comprises nylon, lycra, polyester, or spandex; and wherein the second material is neoprene.
